(19) United States
(12) Patent Application Publication
(10) Pub. No.: US 2010/0189820 A1
(43) Pub. Date: Jul. 29, 2010

(54) Composition and Methods for Maintaining Skeletal Muscle Androgen Receptivity

(75) Inventors: Ken Clement, Oakville (CA); Shan Chaudhuri, Brampton (CA); Michele Molino, Oakville (CA); Phil Apong, Oakville (CA)

Correspondence Address: IOVATE HEALTH SCIENCE RESEARCH INC., 381 North Service Road West, Oakville, ON, L6M 0H4, CA

(73) Assignee: MULTI FORMULATIONS LTD., Oakville (CA)

(21) Appl. No.: 12/119,124

(22) Filed: May 12, 2008

Related U.S. Application Data
(63) Continuation-in-part of application No. 11/746,825, filed on May 10, 2007.

(51) Int. Cl. A61K 36/889

(52) U.S. Cl. 424/727

(57) ABSTRACT

A nutritional composition comprising at least L-carnitine fumarate, red wine extract, and saw palmetto extract is provided to maintain skeletal muscle androgen receptivity, via simultaneous increase in the availability of androgen receptors and improved availability of testosterone, while at the same time reducing some of the adverse effects of increased testosterone levels. A method of same is also provided. The present invention further comprises one or more of N-acetyl L-Carnitine, Ubidecarenone (coenzyme Q10), Idebenone, Decylubiquinone, Astaxanthin, and Ginsenoside Rb1.

### CROSS-REFERENCE TO RELATED APPLICATION

This application is a Continuation-in-Part and claims priority to U.S. patent application Ser. No. 11/746,825, filed May 10, 2007, the entirety of which is incorporated herein by reference.

### FIELD OF THE INVENTION

The present invention relates to a novel and improved nutritional supplement for maintaining skeletal muscle androgen receptivity, via simultaneous increase in the availability of androgen receptors and improved availability of testosterone, while at the same time reducing some of the adverse effects of increased testosterone levels. More specifically, the present invention relates to a composition comprising a combination of L-Carnitine fumarate, red wine extract and saw palmetto extract.

### BACKGROUND OF THE INVENTION

Androgen receptors (AR) are intracellular receptors that specifically bind androgens, such as testosterone and dihydrotestosterone, but are also known to be activated by growth factors, such as insulin-like growth factor-1 (IGF-1). The influence of testosterone on skeletal muscle protein synthesis is mediated by the AR. After an androgen binds to the AR, restructuring and dimerization of the proteins occurs forming an activated receptor complex, which translocates to the nucleus and binds to DNA, thereby activating androgen-specific gene expression in the nucleus.

Animal and clinical studies indicate that the AR signaling pathway is required for the appropriate development of skeletal muscles, as it regulates increases in lean muscle mass, muscle strength, and muscle protein synthesis. The importance of AR for muscle protein accretion has been shown, since muscle hypertrophy has been shown to be attenuated by AR blockade.

In trained individuals, high-volume, high-intensity resistance exercise appears to cause a decrease in AR protein content within 1 hour post-exercise, almost certainly due to protein catabolism induced by exercise-related stress. Accordingly, compositions and methods to maintain muscle AR content and receptivity are desired.

### SUMMARY OF THE INVENTION

The present invention relates to a novel and improved nutritional supplement for maintaining skeletal muscle androgen receptivity, via simultaneous increase in the availability of androgen receptors and improved availability of testosterone, while at the same time reducing some of the adverse effects of increased testosterone levels. The effects of the present composition on increasing and maintaining androgens and androgen-like substances in an individual allows them to act upon an androgen receptor wherein they confer their respective endogenous effects. The nutritional supplement comprising at least an effective amount of L-Carnitine fumarate, red wine extract, and an effective amount of saw palmetto extract. In additional aspects of the present invention, one or more of N-acetyl L-Carnitine, Ubidecarenone (coenzyme Q10), Idebenone, Decylubiquinone, Astaxanthin, and Ginsenoside Rb1 are added to the composition to provide further synergistic benefits. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional supplement, for maintaining skeletal muscle androgen receptivity, via simultaneous increase in the availability of androgen receptors and improved availability of testosterone, while at the same time reducing some of the adverse effects of increased testosterone levels, comprising L-Carnitine fumarate, red wine extract, and saw palmetto extract. According to various aspects, the present invention may further comprise combinations of N-acetyl L-Carnitine, Ubidecarenone (coenzyme Q10), Idebenone, Decylubiquinone, Astaxanthin, and Ginsenoside Rb1.

The term ‘receptivity’ as used herein is understood to define the ability of a cell to interact with an extracellular substance, via membrane proteins, to produce intracellular signals and protein translocation to the nucleus resulting in gene expression. It is herein understood that receptivity is enhanced by factors including, but not limited to the increased presence of extracellular substances, e.g. androgens, increased presence of activated cell receptors as a result of reduced cell damage, enhancements of interactions between substances and receptors, and increased ability to propagate intracellular signal cascades post-receptor-substance interaction within a cell.

A used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to a category of compositions having at least one physiological function when administered to a mammal by conventional routes of administration.

L-Carnitine and Functional Derivatives

L-Carnitine is a quaternary ammonium compound synthesized from the amino acids lysine and methionine. L-Carnitine plays a role in the transport of fatty acids across the mitochondrial matrix for the subsequent metabolism and energy production by beta-oxidation.

However, recent research in this area has mostly involved L-Carnitine L-Tartrate (LCLT), a salt of L-Carnitine, and has focused on a role separate from Carnitine's originally hypothesized role in fat metabolism. LCLT supplementation has been evaluated on resistance exercise trained humans as an enhancer of the hormonal responses to resistance exercise and as a recovery promoter. Three weeks of supplementation with LCLT, providing the equivalent of 2 g of elemental Carnitine per day has been shown to reduce muscle damage produced by an acute bout of high-intensity resistance exercise via cross-over, placebo controlled studies. The investigators conclude that less muscle damage may have resulted in more hormonal receptors available for binding interactions with anabolic hormones. This explains the reduced progression of muscle damage, as measured by MRI, in the recovery days after resistance exercise. One study has shown that 21-days of L-Carnitine supplementation, with 2 g of Carnitine per day, in weight-trained individuals induced significant up-regulation of pre-exercise skeletal muscle AR protein content (p<0.05) as compared to placebo. L-Carnitine confers its function in this regard by reducing muscle damage associated with resistance exercise, therefore attenuating the catabolism of muscle-specific proteins, such as AR, for example. It is understood that L-Carnitine enhances testosterone uptake via offering a protective effect resulting in a reduction in muscle damage and an increased availability of AR. It is understood that L-Carnitine's effects are not conferred via direct stimulation of testosterone secretion. Based on these considerations, and on the fact that post-resistance exercise feeding stimulates increases in AR content, it is herein understood by the inventors that L-Carnitine and feeding independently yet synergistically enhance the hormonal environments following resistance exercise and promote anabolism.

Furthermore, a study of the effects of L-Carnitine supplementation on delayed muscle soreness showed that L-Carnitine has a protective effect against pain and damage from eccentric muscular effort. This result has been attributed to the vasodilative properties of L-Carnitine, which would increase the wash-out of pain inducing energy metabolites.

It is herein understood by the inventors that supplementation with the equivalent of 2 g of L-Carnitine per day, will reduce the catabolism of muscle-specific proteins, resulting in enhanced testosterone uptake via the increased availability of AR. Furthermore, it is understood that the vasodilative properties of the composition will improve energetic metabolism in hypoxic muscle tissue and enhance the wash-out of pain generating energy metabolites, thus decreasing muscle damage and pain and resulting in quicker recovery following resistance exercise.

In an embodiment of the present invention, which is set forth in greater detail in the examples below, the nutritional supplement comprises effective sources of L-Carnitine, such as, but not limited to, L-Carnitine fumarate, L-Carnitine-L-Tartrate, and N-acetyl L-Carnitine HCI. In addition to the aforementioned derivatives, other effective and pharmaceutically acceptable salts or ester of carnitine may be employed in the practice of the invention. By way of example, a serving of the nutritional supplement comprises from about 0.5 g to about 5.0 g of L-Carnitine fumarate, and from about 0.01 g to about 1 g of N-acetyl L-Carnitine. The preferred dosage of the nutritional supplement of the present invention comprises about 1.25 g of L-Carnitine fumarate and about 0.375 g of N-acetyl L-Carnitine per serving.

Red Wine Extract

Red wine extract, Vitus vinifera, is a rich sources of flavonoids, known for their strong antioxidant properties; and important nonflavonoids such as resveratrol, which is a phytoalexin produced by some stressed plants. A study using breast cancer cells transfected with aromatase, a viable model for screening aromatase inhibitors, showed that resveratrol inhibits aromatase activity. Additionally, the same study showed that resveratrol was shown to reduce the abundance of aromatase mRNA.

One of the most abundant flavonoids from red wine is procyanidin. Procyanidins are essentially polymer chains of flavonoids that have been shown to be potent inhibitors of the action of aromatase. Procyanidin's inhibitory action on aromatase is due to its competition with the binding of androgen substrates. This inhibition of the human aromatase enzyme inhibits the conversion of testosterone to estradiol resulting in increased levels of androgens, e.g. testosterone, in the body.

It is herein understood by the inventors that compositions comprising red wine extract will act to increase levels of free androgens in the body by reducing the conversion of active androgens, such as testosterone, into inactive substances, such as estrodiols. By inhibiting aromatase enzymes, red wine extract will facilitate more numerous interactions of testosterone with AR, leading to increases in lean muscle mass, muscle strength and muscle protein synthesis.

In an embodiment of the present invention, which is set forth in greater detail in the examples below, the nutritional supplement comprises red wine extract. A serving of the nutritional supplement comprises from about 0.001 g to about 0.100 g of red wine extract. The preferred dosage of a serving of the nutritional supplement comprises about 0.025 g of red wine extract.

Saw Palmetto (Serenoa repens or Sabal serrulata)

Saw palmetto is a fruit extract that is rich in fatty acids and phytosterols. The berries of saw palmetto contain fatty acids which include: caprylic, lauric, caproic, oleic, capric and palmitic acids, and esters thereof. Saw palmetto has been used throughout history by Native Americans, the Mayans and European colonists for various conditions such as, asthenia, recovery from major illness and urogenital problems. In modern times, lipophilic extracts of Serenoa repens are most commonly used to treat benign prostatic hyperplasia (BPH), as well as potentially treating male-pattern baldness.

Comparison of saw palmetto versus finasteride, a drug used to treat BPH, showed similar efficacy for suppressing DHT levels, in both cases, as compared to placebo. It has been purported that the efficacy of saw palmetto in reducing symptoms of BPH are due to its inhibition of 5α-reductase. 5α-reductase is the enzyme responsible for the conversion of testosterone to the more problematic androgen, dihydrotestosterone (DHT). DHT is one of the primary contributors to male-pattern baldness, and plays a role in the development or exacerbation BPH and prostate cancer.

It is herein understood by the inventors that compositions comprising saw palmetto extract will act to increase levels of testosterone in the body by reducing its conversion to DHT. By inhibiting 5α-reductase, saw palmetto extract will reduce the increases in DHT that often accompanies increases in free testosterone, and thereby act to reduce some of the adverse effects that often accompany increased levels of DHT, such as but not limited to, male-pattern baldness and BPH.

In an embodiment of the present invention, which is set forth in greater detail in the examples below, the nutritional supplement comprises saw palmetto extract. A serving of the nutritional supplement comprises from about 0.005 g to about 0.500 g of saw palmetto extract. The preferred dosage of a serving of the nutritional supplement comprises about 0.200 g of saw palmetto extract.

Simultaneous Increase in Androgen Receptors and Androgens

The present invention is comprised of components for enhancing hormonal responses to resistance exercise; reducing muscle damage associated with resistance exercise; producing protective effects against pain and damage from eccentric effort by acting as a vasodilator; and increasing levels of free androgens in the body, all while mitigating some of the adverse effects associated with increased androgen levels.

According to one embodiment of the invention, the composition comprises at least L-Carnitine fumarate, red wine extract, and saw palmetto extract, wherein the L-Carnitine fumarate will reduce the catabolism of muscle-specific proteins, resulting in enhanced testosterone uptake via the increased availability of AR; the use of red wine extract will competitively inhibit the binding of androgen substrates to aromatase enzymes thereby reducing the conversion of testosterone to estrodiol; and the saw palmetto extract will inhibit 5α-reductase conversion of testosterone to DHT. Thus increased levels of free testosterone and reduction of the adverse effects that accompany increased testosterone level, along with the enhanced testosterone uptake will result in greater stimulation of the AR signaling pathway, leading to increases in lean muscle mass, muscle strength, and muscle protein synthesis.

It is herein understood by the inventors that the above combination of ingredients work substantially simultaneously and synergistically to help mediate a faster recovery following resistance exercise, reduce the catabolism of muscle-specific proteins, resulting in enhanced testosterone uptake via increased availability of AR than is normally observed in the absence of such supplementation, as well as reduction in adverse effects that often accompany increased levels of testosterone.

In additional aspects of the present invention, one or more of N-acetyl L-Carnitine, Ubidecarenone (coenzyme Q10), Idebenone, Decylubiquinone, Astaxanthin, and Ginsenoside Rb1 are added to the composition to provide further synergistic benefits relating to maintaining skeletal muscle androgen receptivity. The additional ingredients and synergistic benefits are disclosed hereinafter.

Ubidecarenone (Coenzyme Q10), Idebenone and Decylubiquinone

Coenzyme Q10 (CoQ10, Ubidecarenone) is found in the mitochondria of all cells and is involved in energy production. It is found at its highest concentrations in the heart, liver, kidney and pancreas. CoQ10 is a potent antioxidant in human blood where it also acts to preserve vitamin E, another major antioxidant. As a result of CoQ10's antioxidant activity it exerts a protective effect on mitochondrial membranes, insuring the integrity of the membrane-receptor interface.

One study has shown that individuals suffering from angina were able to exercise for longer periods when receiving CoQ10 as compared to untreated groups. Moreover, myocardial function was improved by CoQ10 in patients with disease conditions known to involve energy production deficits wherein these patients also reported a ‘subjective’ improved sense of well-being.

Idebenone and Decylubiquinone are synthetic CoQ10 derivatives, the former being a potent antioxidant with the ability to fight reactive oxygen species (ROS) under low oxygen tension situations. As a result of this inhibition of lipid peroxidation, Idebenone acts to protect cell membranes, especially those of the mitochondria, from oxidative damage. Decylubiquinone has been shown to effectively block redox-dependent mitochondrial permeability transition, thereby reducing the loss of mitochondrial transmembrane potential.

It is herein understood by the inventors that compositions comprising one or more of these antioxidant quinones exert a protective effect on mitochondrial membranes, thus ensuring the integrity of the membrane-receptor interface and preserving the effect of hormones on mitochondrial cholesterol transport and steroidogenesis.

In various embodiments of the present invention, which are set forth in greater detail in the examples below, the nutritional supplement comprises Ubidecarenone (Coenzyme Q10), Idebenone, Decylubiquinone or combinations thereof. A serving of the nutritional supplement comprises from about 0.0001 g to about 0.01 g of Ubidecarenone (Coenzyme Q10), from about 0.00001 g to about 0.01 g of Idebenone, from about 0.000001 g to about 0.0001 g of Decylubiquinone, or combinations thereof. The preferred dosage of a serving of the nutritional supplement of the present invention comprises about 0.001 g of Ubidecarenone (Coenzyme Q10), and about 0.001 g of Idebenone.

Ginsenoside Rb1

Ginsenosides are a class of steroid-like compounds, found exclusively in plants, Panax quinquefoius. Ginsenosides have been the target of research, since they are viewed as the active compounds behind the claims of ginseng's efficacy. Ginsenosides appear to affect multiple pathways, and so their effects are complex and difficult to isolate.

Ginsenoside Rb1 has been shown in animals to stimulate the secretion of lutenizing hormone (LH) after exercise. Lutenizing hormone is synthesized and secreted by the anterior pituitary gland and is responsible for the stimulation of Leydig cell production of testosterone. It is herein understood by the inventors that increased secretion of LH will result in a greater production of testosterone, thus greater levels of serum testosterone leading to more numerous interactions of testosterone with AR. This will lead to increases in lean muscle mass, muscle strength, and muscle protein synthesis.

In an embodiment of the present invention, which is set forth in greater detail in the examples below, the nutritional supplement comprises Ginsenoside Rb1. A serving of the nutritional supplement comprises from about 0.000001 g to about 0.0001 g of Ginsenoside Rb1. The preferred dosage of a serving of the nutritional supplement comprises about 0.00005 g of Ginsenoside Rb1.

In an embodiment of the present invention, which is set for in greater detail in Example 1, the nutritional supplement comprises L-Carnitine fumarate, red wine extract, and saw palmetto extract. The composition is provided in any acceptable and suitable oral dosage from as known in the art to maintain skeletal muscle androgen receptivity and reduce some of the adverse effects associated with increased testosterone levels.

In another embodiment of the present invention, which is set for in greater detail in Example 2, the nutritional supplement comprises L-Carnitine fumarate, red wine extract, saw palmetto extract, N-acetyl L-Carinitine, Ginsenoside Rb1, Idebenone, Coleus foskohlii extract, Coenzyme Q10, Astaxanthin, Eurycoma longifolia, and Yohimbine. The composition is provided in any acceptable and suitable oral dosage from as known in the art to maintain skeletal muscle androgen receptivity and reduce some of the adverse effects associated with increased testosterone levels.

In an embodiment of the present invention, which is set forth in greater detail Example 3, the nutritional supplement comprises L-Carnitine fumarate and an extract of Coleus forskohlii. The composition is provided in any acceptable and suitable oral dosage form as known in the art to maintain androgen uptake potential of cells, and minimize muscle damage associated with resistance exercise.

In another embodiment of the present invention, which is set forth in greater detail Example 4, the nutritional supplement comprises L-Carnitine fumarate, an extract of Coleus forskohlii, N-acetyl L-Carnitine, melatonin, Coenzyme Q10, Idebenone, Decylubiquinone, an extract of Agaricus blazei murill, and Ginsenoside Rb1. The composition is provided in any acceptable and suitable oral dosage form as known in the art to maintain androgen uptake potential of cells, via simultaneous increase in the availability of androgen receptors and improved availability of androgen molecules, as well as to minimize muscle damage associated with resistance exercise.

### Additional Embodiments for Maintaining Skeletal Muscle Androgen Receptivity

The present invention is comprised of components that have been shown to enhance hormonal responses to resistance exercise, reduce muscle damage associated with resistance exercise and produce a protective effect against pain and damage from eccentric effort by acting as a vasodilator, all while reducing some of the adverse effects associated with increased androgen levels. It is herein understood by the inventors that inclusion of L-Carnitine fumarate in the claimed composition will act to facilitate a faster recovery following resistance exercise, reduce the catabolism of muscle-specific proteins, resulting in enhanced testosterone uptake via increased availability of AR than is normally observed in the absence of such supplementation. Furthermore, it is understood that the vasodilative properties provided by the L-Carnitine fumarate will act to improve the energetic metabolism of the hypoxic muscle and enhance the wash-out of pain generating metabolites.

Additionally, the present invention comprises red wine extract which has been shown to competitively inhibit the binding of androgen substrates to aromatase enzymes thereby reducing the conversion of testosterone to estrodiol. It is herein understood by the inventors that reduction of testosterone conversion will lead to increases in lean muscle mass, muscle strength, and muscle protein synthesis.

Additionally, the present invention comprises saw palmetto extract which has been shown to inhibit 5α-reductase activity, thereby reducing the conversion of testosterone to DHT. It is herein understood by the inventors that reduction in levels of DHT will lead to increases in lean muscle mass, muscle strength, and muscle protein synthesis, as well as reduce some of the adverse effects associated with increased levels of testosterone.

In addition, the present invention may additionally comprise Ubidecarenone (Coenzyme Q10) and/or derivatives thereof, which exhibit antioxidant activities and have protective effects on mitochondrial membranes. It is herein understood by the inventors that these protective effects will ensure the integrity of the membrane-receptor interface and preserve the effect of hormones on mitochondrial cholesterol transport and steroidogenesis.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is as a softgel.

Furthermore, the dosage form of the nutritional supplement may be provided in accordance with customary processing techniques for herbal and nutritional supplements in any of the forms mentioned above. Additionally, the nutritional supplement set forth in the example embodiments herein disclosed may contain any appropriate number and type of excipients, carriers or additional ingredients, as is well known in the art.

By way of ingestion of the composition of the present invention, a method for substantially simultaneously reducing the catabolism of muscle-specific proteins as well as inhibiting the degradation of testosterone while mitigating adverse effects associated with increased testosterone levels is provided. The increase of androgens levels resulting from the method of the present invention then confer their respective actions on androgen receptors. The method of the present invention comprises at least the step of administering to an individual a therapeutically acceptable amount of the composition of the present invention.

Although the following examples illustrate the practice of the present invention in two of its embodiments, the examples should not be construed as limiting the scope of the invention. Other embodiments will be apparent to one of skill in the art from consideration of the specifications and example.

### EXAMPLES

### Example 1

A nutritional supplement comprising the following ingredients per serving is prepared for consumption as three softgels to be consumed twice daily:
About 1.25 g of L-Carnitine fumarate, about 0.025 g of red wine extract, and about 0.200 g of saw palmetto extract.
Preferably, the nutritional supplement is consumed in accordance with the following directions:
Directions: The supplement should be consumed in 2 servings per day, one taken with a main meal and the other with a pre-workout meal (generally consumed 1 hour prior to commencement of exercise). On non-workout days, the supplement should be taken with main meals, one of which is dinner. Supplementation should last for a 21 day cycle, with a 7 to 10 day wash-out period before commencement of another treatment round.

### Example 2

A nutritional supplement comprising the following ingredients per serving is prepared for consumption as a caplet to be consumed twice daily:
About 1.25 g of L-Carnitine fumarate, about 0.025 g of red wine extract, and about 0.20 g of saw palmetto extract, about 0.375 g of N-acetyl L-Carnitine, about 0.00005 g of ginsenoside Rb1, about 0.001 g of Idebenone, about 0.025 g of an extract of Coleus forskohlii standardized to 10% forskolin, about 0.001 g of Ubidecarenone (coenzyme Q10), about 0.15 g of Astaxanthin, about 0.001 g of Eurycoma longifolia (whole herb), and about 0.003 g of Yohimbine HCI. Preferably, the nutritional supplement is consumed in accordance with the following directions:
Directions: The supplement should be consumed in 2 servings per day, one taken with a main meal and the other with a pre-workout meal (generally consumed 1 hr prior to commencement of exercise). On non-workout days, the supplement should be taken with main meals, one of which is dinner. Supplementation should last for a 21 day cycle, with a 7 to 10 day wash-out period before commencement of another treatment round.

### Example 3

A nutritional supplement comprising the following ingredients per serving is prepared for consumption as a caplet to be consumed twice daily:
About 1.25 g of L-carnitine fumarate and about 0.025 g of an extract of Coleus forskohlii standardized to 10% forskolin.
Preferably, the nutritional supplement is consumed in accordance with the following directions:
Directions: The supplement should be consumed in 2 servings per day, one taken with a main meal and the other with a pre-workout meal (generally consumed 1 hour prior to commencement of exercise). On non-workout days, the supplement should be taken with main meals, one of which is dinner. Supplementation should last for a 21 day cycle, with a 7 to 10 day wash-out period before commencement of another treatment round.

### Example 4

A nutritional supplement comprising the following ingredients per serving is prepared for consumption as a caplet to be consumed twice daily:
About 1.25 g of L-carnitine fumarate, about 0.025 g of an extract of Coleus forskohlii standardized to 10% forskolin, about 0.375 g of N-acetyl L-carnitine, about 0.0005 g of melatonin, about 0.001 g of ubidecarenone (coenzyme Q10), about 0.001 g of Idebenone, about 0.00001 g of dexylubiquinone, about 0.001 g of an extract of Agaricus blazei Murill, and about 0.00005 g of ginsenoside Rb1. Preferably, the nutritional supplement is consumed in accordance with the following directions:
Directions: The supplement should be consumed in 2 servings per day, one taken with a main meal and the other with a pre-workout meal (generally consumed 1 hour prior to commencement of exercise). On non-workout days, the supplement should be taken with main meals, one of which is dinner. Supplementation should last for a 21 day cycle, with a 7 to 10 day wash-out period before commencement of another treatment round.

### Example 5

A nutritional supplement comprising the following ingredients per serving is prepared for consumption as a caplet to be consumed twice daily:
About 1.125 g of L-carnitine fumarate, about 0.0001 g of idebenone, about 0.00001 g of decylubiquinone, about 0.001 g of an extract of Agaricus blazei murill, and about 0.00005 g of ginsenoside Rb1.
Preferably, the nutritional supplement is consumed in accordance with the following directions:
Directions: The supplement should be consumed in 2 servings per day, one taken with a main meal and the other with a pre-workout meal (generally consumed 1 hour prior to commencement of exercise). On non-workout days, the supplement should be taken with main meals, one of which is dinner. Supplementation should last for a 21 day cycle, with a 7 to 10 day wash-out period before commencement of another treatment round.

### Extensions and Alternatives

In the foregoing specification, the invention has been described with specific embodiments thereof; however, it will be evident that various modifications and changes may be made thereto without departing from the broader spirit and scope of the invention.

What is claimed:

1. An orally administrable nutritional composition comprising:
from about 0.50 g to about 5.00 g of L-Carnitine fumarate;
from about 0.001 g to about 0.100 g of red wine extract; and
from about 0.005 g to about 0.500 g of saw palmetto extract.

2. The composition of claim 1 wherein the amount of L-Carnitine fumarate is 1.25 g; the amount of red wine extract is 0.025 g; and the amount of saw palmetto extract is 0.200 g.

3. The composition of claim 1, wherein the composition is provided to a mammal in need thereof in an acceptable oral dosage format.

4. The composition of claim 3, wherein the acceptable oral dosage format is selected from the group consisting of tablets, caplets, beads, powder, capsules, and softgels.

5. The composition of claim 3, wherein the acceptable oral dosage format is a softgel.

6. A method for maintaining skeletal muscle androgen receptivity comprising the step of administering to a mammal a composition comprising:
from about 0.050 g to about 5.00 g of L-Carnitine fumarate;
from about 0.001 g to about 0.100 g of red wine extract; and
from about 0.005 g to about 0.500 g of saw palmetto extract.

7. The method of claim 6 wherein the amount of L-Carnitine fumarate is 1.25 g; the amount of red wine extract is 0.025 g; and the amount of saw palmetto extract is 0.200 g.

8. The method of claim 6, wherein the composition is provided to a mammal in need thereof in an acceptable oral dosage format.

9. The method of claim 8, wherein the acceptable oral dosage format is selected from the group consisting of tablets, caplets, beads, powder, capsules, and softgels.

10. The method of claim 8, wherein the acceptable oral dosage format is a softgel.
